(19) United States
(12) Patent Application Publication
(10) Pub. No.: US 2010/0185295 A1
(43) Pub. Date: Jul. 22, 2010

(54) ARTICULATION PROSTHESIS FOR SMALL BONES, IN PARTICULAR FOR PHALANGEAL, METACARPO-PHALANGEAL OR METATARSO-PHALANGEAL ARTICULATIONS

(75) Inventor: Mohan Emmanuel, Station Town Durham (GB)

Correspondence Address: YOUNG & THOMPSON, 209 Madison Street, Suite 500, Alexandria, VA, 22314, US

(73) Assignee: IMPLANTS INTERNATIONAL LIMITED, Cleveland Thornaby-on-tees (GB)

(21) Appl. No.: 12/306,406

(22) PCT Filed: Jun. 25, 2007
(86) PCT No.: PCT/IB07/01735

§ 371 Date: Jan. 11, 2010

Related U.S. Application Data
(60) Provisional application No. 60/815,867, filed on Jun. 23, 2006.

(30) Foreign Application Priority Data
Jul. 18, 2006 (FR) 06 06503

(51) Int. Cl. A61F 2/42

(52) U.S. Cl. 623/21.11

(57) ABSTRACT

A prosthesis includes a bone anchoring element and a meniscal element capable of being installed on this anchoring element which includes a medullary anchor shaft with a hollow interior, defining a cavity, and a transverse rod fixed and extending through this cavity, and the meniscal element includes an assembly shaft intended to be engaged in the cavity, this assembly shaft having a longitudinal slot having a width smaller than the transverse section of the transverse rod, one end of which opens in the free end of the assembly shaft and the other end of which opens in a transverse hole having a cross-section slightly larger than that of the transverse rod, the assembly shaft intended, thanks to this slot, to be engaged on the transverse rod until engagement of the transverse rod in the transverse hole, with locking, when the meniscal element is in its assembled position on the bone anchoring element.

The present invention relates to a joint prosthesis for small bones, particularly for phalangeal, metacarpo-phalangeal or metatarso-phalangeal joints.

A joint prosthesis for small bones is known, comprising a first anchoring element in one of the bones, forming a joint surface, a second anchoring element in the other bone and a meniscal element able to be installed on this second anchoring element.

The dimensions of the bone anchoring elements were reduced. The problem exists for this type of prosthesis of obtaining reliable installation of the meniscal element on the corresponding bone anchoring element, and to be able to carry out this assembly easily and quickly.

The existing prostheses are not completely satisfactory from this point of view, and the primary objective of the invention is to resolve this drawback.

Moreover, existing prostheses do not always make it possible to easily place the meniscal element in a set position relative to the bone anchoring element intended to receive it.

The invention also has the objective of proposing a prosthesis resolving this drawback.

The prosthesis concerned comprises, in a known manner, a first anchoring element in one of the bones, forming a joint surface, a second anchoring element in the other bone and a meniscal element capable of being installed on this second anchoring element.

According to the invention,
said second bone anchoring element comprises a medullary anchor shaft with a hollow interior, defining a cavity, and a transverse rod fixed such that it extends through this cavity, and
the meniscal element comprises an assembly shaft intended to be engaged in said cavity, this assembly shaft having a longitudinal slot having a width smaller than the transverse section of said transverse rod, one end of which opens in the free end of the assembly shaft and the other end of which opens in a transverse hole having a cross-section slightly larger than that of said transverse rod, said assembly shaft being intended, thanks to this slot, to be engaged on said transverse rod until engagement of said transverse rod in said transverse hole, with locking, when the meniscal element is in its assembled position on said second bone anchoring element.

The meniscal element of the prosthesis according to the invention thus comprises a slotted assembly shaft, defining two parallel distal portions, and a receiving hole with locking of the corresponding transverse rod of the bone anchoring element. In practice, upon insertion of the assembly shaft in the cavity of the bone anchoring element, said parallel distal portions encounter the transverse rod, are elastically distanced from each other by insertion of this transverse rod between them and then, when the transverse rod penetrates said transverse hole, resume their initial position, ensuring retention of the transverse rod in the transverse hole and, because of this, assembly of the meniscal element in this set position.

When the meniscal element must be placed in a set position relative to the anchoring element, in particular when it comprises a rib or groove for guiding the movement of said first anchoring element, said transverse rod and said slot make it possible to place the meniscal element in this set position easily.

If necessary, the assembly shaft of the meniscal element comprises one or several recesses or flats at the level of at least one of the openings through which said transverse hole opens to the outside of this assembly shaft.

This or these recess(es) or flat(s) make it possible to reduce the cross-section of the assembly shaft in this place in order to increase the flexibility of said parallel distal portions defined by the slot.

Said medullary anchoring shaft of said second bone anchoring element and said assembly shaft of the meniscal element may have a tapered shape.

Said second anchoring element may have a collar for receiving the meniscal element. This collar may be connected to said medullary anchoring shaft by a flared portion, and said transverse rod may be located immediately below this flared portion.

The invention shall be clearly understood, and other characteristics and advantages thereof shall appear, in reference to the annexed diagrammatic drawing, illustrating, as a non-exhaustive example, a preferred embodiment of a bone anchoring element and of a meniscal element comprised by the prosthesis in question.

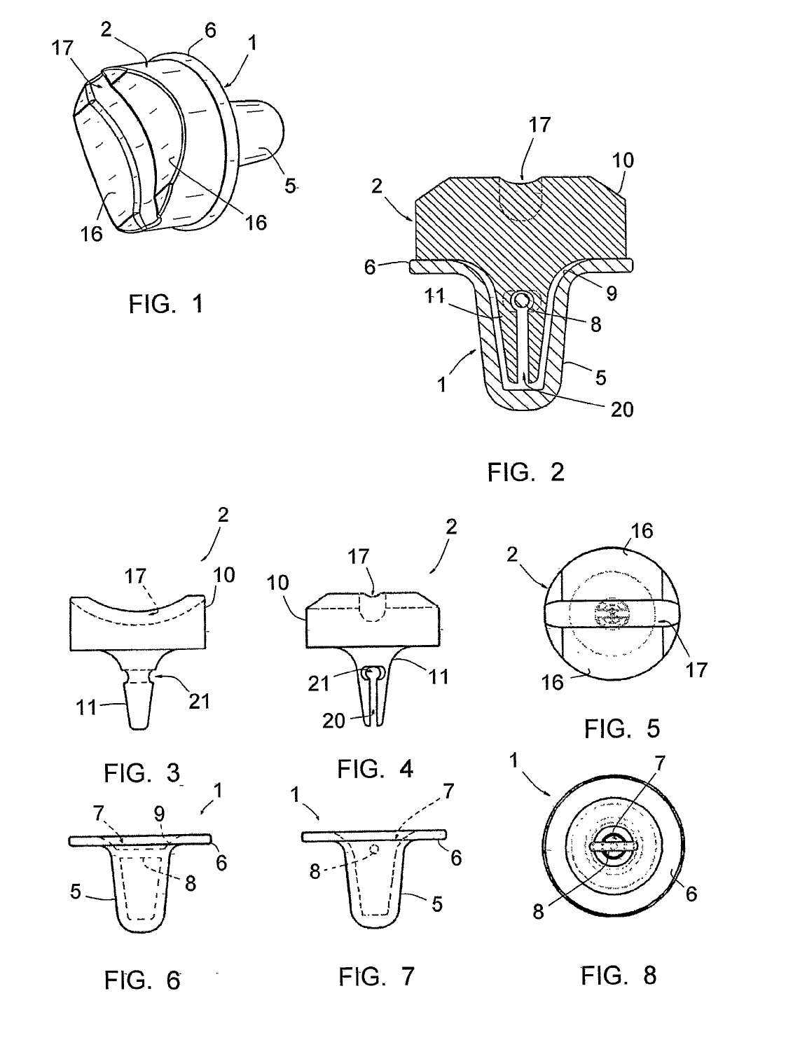

FIG. 1 is a perspective view of this bone anchoring element and the meniscal element assembled on said bone anchoring element;

FIG. 2 is a cross-section of these elements following the axis of these elements;

FIGS. 3 to 5 are views of the meniscal elements from a side according to a first direction, a side according to a second direction, perpendicular to this first direction, and from below, respectively, and

FIGS. 6 to 8 are views of the bone anchoring element from a side according to a first direction, a side according to a second direction, perpendicular to this first direction, and from below, respectively.

The figures illustrate an anchoring element 1 and a meniscal element 2 which are part of a joint prosthesis for small bones, particularly for phalangeal, metacarpo-phalangeal and metatarso-phalangeal joints.

The anchoring element 1 is intended to be anchored in one of the bones forming the joint to be treated, and the prosthesis also comprises another bone anchoring element (not shown) intended to be anchored in the other bone forming the joint to be treated. This other bone anchoring element comprises a joint surface intended to cooperate with a joint surface formed by the meniscal element 2.

As shown in FIGS. 2 and 6 to 8, the anchoring element 1 comprises a medullary anchoring shaft 5 and a collar 6. The assembly is formed by a single piece of an appropriate metallic material, particularly in a chrome-cobalt alloy.

The medullary anchor shaft 5 is tapered in shape and is hollow inside, defining a cavity 7, and comprises a transverse rod 8 fixed to it, extending through this cavity 7. This medullary anchor shaft 5 is connected to the collar 6 by a flared portion 9 and the transverse rod 8 is located immediately below this flared portion 9.

The meniscal element 2 comprises a part 10 in the shape of a plateau and an assembly shaft 11. The assembly is formed by a piece of a material promoting sliding, for example in high-density polyethylene.

The part 10 forms said joint surface; in the illustrated example, this joint surface presents lateral glenoid surfaces 16 and a median guide groove 17, intended to cooperate with lateral condyle surfaces and with a median rib comprised by the joint surface of said other bone anchoring element, respectively. The part 10 also forms, from the side of the assembly shaft 11, a flat surface intended to be received on the collar 6.

The assembly shaft 11 is tapered in shape and connected to the part 10 by a flared area. It presents one longitudinal slot 20 having a width smaller than that of the transverse section of the transverse rod 8, one end of which opens in the free end of the assembly shaft 11 and the other end of which opens in a transverse hole 21 having a cross-section slightly larger than that of said transverse rod 8. The assembly shaft 11 also comprises two flats at the level of at least one of the openings through which the hole 21 opens to the outside of this assembly shaft.

As can be seen in reference to FIG. 2, the meniscal element 2 is intended to be assembled on the anchoring element 1 by engagement of the assembly shaft 11 in the cavity 7 of the anchor shaft 5. The assembly shaft 11 is intended, thanks to the slot 20, to be engaged on the transverse rod 8 until engagement of this rod 8 in the hole 21, with locking, when the meniscal element 2 is in the assembled position on the anchor element 1. In practice, during insertion of the assembly shaft 11 in the cavity 7, the parallel distal portions of this shaft 11 defined by the slot 20 come into contact with the transverse rod 8, are elastically distanced from each other by insertion of this rod 8 between them then, when the rod 8 penetrates the hole 21, resume their initial position, ensuring retention of the rod 8 in the hole 21 and, because of this, assembly of the meniscal element 2 on the anchor element 1.

As shown by the preceding, the invention provides a joint prosthesis presenting determining advantages to enable reliable assembly of the meniscal element on the bone anchoring element, able to be performed easily and quickly, and to allow easy placement of the meniscal element in a set position relative to the anchoring element.

It goes without saying that the invention is not limited to the embodiment described above as an example, but that it extends to all embodiments covered by the annexed claims.

1. Joint prosthesis for small bones, particularly for phalangeal, metacarpo-phalangeal and metatarso-phalangeal joints, comprising a first anchoring element in one of the bones, forming a joint surface, a second anchoring element (1) in the other bone and a meniscal element (2) capable of being installed on this second anchoring element (1), characterized in that:
said second bone anchoring element (1) comprises a medullary anchor shaft (5) with a hollow interior, defining a cavity (7), and a transverse rod (8) fixed such that it extends through this cavity (7), and
the meniscal element (2) comprises an assembly shaft (11) intended to be engaged in said cavity (7), this assembly shaft (11) having a longitudinal slot (20) having a width smaller than the transverse section of said transverse rod (8), one end of which opens in the free end of the assembly shaft (11) and the other end of which opens in a transverse hole (21) having a cross-section slightly larger than that of said transverse rod (8), said assembly shaft (11) being intended, thanks to this slot (20), to be engaged on said transverse rod (8) until engagement of said transverse rod (8) in said transverse hole (21), with locking, when the meniscal element (2) is in its assembled position on said second bone anchoring element (1).

2. Prosthesis according to claim 1, characterized in that the meniscal element (2) comprises a rib or groove (17) for guiding the movement of said first anchoring element (1)

3. Prosthesis according to claim 1, characterized in that the assembly shaft (11) of the meniscal element (2) comprises one or several recesses or flats at the level of at least one of the openings through which said transverse hole (21) opens to the outside of this assembly shaft (11).

4. Prosthesis according to claim 1, characterized in that said medullary anchoring shaft (5) of said second bone anchoring element (1) and said assembly shaft (11) of the meniscal element (2) have a tapered shape.

5. Prosthesis according to claim 1, characterized in that said second anchoring element (1) has a collar (6) for receiving the meniscal element (2).

6. Prosthesis according to claim 5, characterized in that said collar (6) is connected to said medullary anchoring shaft (5) by a flared portion (9), and in that said transverse rod (8) is located immediately below this flared portion (9).

7. Prosthesis according to claim 2, characterized in that said medullary anchoring shaft (5) of said second bone anchoring element (1) and said assembly shaft (11) of the meniscal element (2) have a tapered shape.

8. Prosthesis according to claim 3, characterized in that said medullary anchoring shaft (5) of said second bone anchoring element (1) and said assembly shaft (11) of the meniscal element (2) have a tapered shape.

9. Prosthesis according to claim 2, characterized in that said second anchoring element (1) has a collar (6) for receiving the meniscal element (2).

10. Prosthesis according to claim 3, characterized in that said second anchoring element (1) has a collar (6) for receiving the meniscal element (2).

11. Prosthesis according to claim 4, characterized in that said second anchoring element (1) has a collar (6) for receiving the meniscal element (2).

12. Prosthesis according to claim 9, characterized in that said collar (6) is connected to said medullary anchoring shaft (5) by a flared portion (9), and in that said transverse rod (8) is located immediately below this flared portion (9).

13. Prosthesis according to claim 10, characterized in that said collar (6) is connected to said medullary anchoring shaft (5) by a flared portion (9), and in that said transverse rod (8) is located immediately below this flared portion (9).

14. Prosthesis according to claim 11, characterized in that said collar (6) is connected to said medullary anchoring shaft (5) by a flared portion (9), and in that said transverse rod (8) is located immediately below this flared portion (9).
